(19) United States
(12) Patent Application Publication
(10) Pub. No.: US 2014/0194499 A1
(43) Pub. Date: Jul. 10, 2014

(54) Polyprenylated Benzoylphloroglucinols With Anticancer Activity

(71) Applicant: Hong Kong Baptist University, Hong Kong (HK)

(72) Inventors: Hongxi XU, HK (HK); Hong ZHANG, HK (HK); Yuanzhi LAO, HK (HK); Dandan ZHANG, HK (HK); Kaixian CHEN, HK (HK); Zhaoxiang BIAN, HK (HK); Chengyuan LIN, HK (HK); Dajian YANG, HK (HK); Shilin CHEN, HK (HK); Aiping LU, HK (HK); Albert Sun Chi CHAN, HK (HK)

(73) Assignee: Hong Kong Baptist University, Hong Kong (HK)

(21) Appl. No.: 14/151,791

(22) Filed: Jan. 9, 2014

Related U.S. Application Data
(60) Provisional application No. 61/750,937, filed on Jan. 10, 2013.

(51) Int. Cl. C07D 311/78; C07C 50/38

(52) U.S. Cl. CPC C07D 311/78; C07C 50/38
USPC 514/454; 549/386; 568/327; 514/681

(57) ABSTRACT

Two novel polyprenylated benzoylphloroglucinol derivatives, garciesculentones A and C, are disclosed in the present invention. Garciesculentones A and C exhibit cytotoxicity and/or antimigration effect against various cancer cell lines. The experimental results of the present invention suggest that Garciesculentones A and C are useful for developing into anticancer drugs.

### CROSS-REFERENCE TO RELATED APPLICATIONS

The present application claims the benefit of U.S. Provisional Patent Application Ser. No. 61/750,937 filed on Jan. 10, 2013, which is hereby incorporated by reference in its entirety.

### FIELD OF INVENTION

The present invention is in the field of pharmaceuticals and chemical industries. In particular, The present invention relates to new chemical entities of Garcinia esculenta Y. H. Li having cytotoxicity against a panel of human cancer cell lines. In particular, the present invention relates to two novel polyprenylated benzoylphloroglucinol derivatives, garciesculentones A and C, which exhibit cytotoxicity and antimigration effect against various human cancer cell lines. The present invention also relates to the use of garciesculentones A and C as active compounds in anticancer drugs.

### BACKGROUND OF INVENTION

Nature has played a dominant role in anticancer drug discovery and development, and over 70% of the anticancer drugs currently available may be classified as natural-product inspired. The genus Garcinia are a rich source of compounds with diverse chemical structures, including xanthones, polyprenylated benzophenones, bioflavonoids, etc., which have been reported in Ting Kan, W. L.; Yin, C.; Xu, H. X.; Xu, G.; To, K. K.; Cho, C. H.; Rudd, J. A.; Lin, G., Anti-tumor effects of novel compound, guttiferone K, on colon cancer by p21Waf1/Cip1-mediated G(0)/G(1) cell cycle arrest and apoptosis. Int J Cancer 2012; (b) Xu, G.; Kan, W. L.; Zhou, Y.; Song, J. Z.; Han, Q. B.; Qiao, C. F.; Cho, C. H.; Rudd, J. A.; Lin, G.; Xu, H. X., Cytotoxic acylphloroglucinol derivatives from the twigs of Garcinia cowa. J Nat Prod 2010, 73 (2), 104-8; (c) Prasad, S.; Ravindran, J.; Sung, B.; Pandey, M. K.; Aggarwal, B. B., Garcinol potentiates TRAIL-induced apoptosis through modulation of death receptors and antiapoptotic proteins. Mol Cancer Ther 2010, 9 (4), 856-68; (d) Wang, X.; Chen, Y.; Han, Q.-b.; Chan, C.-y.; Wang, H.; Liu, Z.; Cheng, C. H.-k.; Yew, D. T.; Lin, M. C. M.; He, M.-I.; Xu, H.-x.; Sung, J. J. Y.; Kung, H.-f., Proteomic identification of molecular targets of gambogic acid: Role of stathmin in hepatocellular carcinoma. PROTEOMICS 2009, 9 (2), 242-253, to possess a wide range of biological activities, especially antitumor effects. Therefore, our research group has been focusing on the isolation of new natural agents with antitumor activities from more Garcinia species and the study on their action mechanism to find potential candidates for anticancer treatment.

Although in prior arts such as U.S. Pat. No. 7,402,706 and US 20110301233 compositions containing polyisopreneylated benzophenones with anticancer activity isolated from Garcinia plants have been disclosed, they are structurally and functionally different from the novel compounds disclosed in the present invention. Furthermore, none of these prior arts disclosed the bioactivities in the novel compounds of our invention. For example, in U.S. Pat. No. 7,402,706 the claimed composition/compound only showed inhibitory effect on histone acetyltransferases p300 and PCAF but no cytotoxicity and antimigration effect on various cancer cells were found in the composition/compound of '706; in US 20110301233, although the disclosed composition/compound showed cytotoxicity against cancer cells, no antimigration effect has been shown. In contrast, the compounds disclosed in the present invention are shown to have both cytotoxicity and antimigration effect on various cancer cell types being tested.

Citation or identification of any reference in this section or any other section of this application shall not be construed as an admission that such reference is available as prior art for the present application.

### SUMMARY OF INVENTION

Accordingly, the objective of the present invention is to provide two new polyprenylated benzoylphloroglucinol derivatives, garciesculentones A and C (represented by chemical structures 1 and 2 in FIG. 1), which exhibit cytotoxicity and antimigration effect against various cancer cell lines and have potential to be developed as anticancer drugs.

In accordance with one aspect of the present invention, there is provided two novel polyprenylated benzoylphloroglucinol derivatives, garciesculentones A and C (represented by chemical structures 1 and 2 in FIG. 1, respectively), which exhibit cytotoxicity against the various cancer cell lines and can be used as active compounds in drugs for cancer treatment.

In accordance with another aspect of the present invention, there is provided two novel polyprenylated benzoylphloroglucinol derivatives, garciesculentones A and C (represented by chemical structures 1 and 2 in FIG. 1, respectively), which exhibit antimigration effect on various cancer cell lines without cytotoxicity and can be used as active compounds in drugs for cancer treatment. The cancer cell lines which the presently claimed compounds exhibits cytotoxicity against and/or antimigration effect on include but not limited to human hepatocellular carcinoma, breast adenocarcinoma and esophageal carcinoma. The anticancer drugs may be administered orally, and/or through intravenous or intraperitoneal injection, to a subject in needs thereof.

A further aspect of the present invention relates to a method for extracting the two polyprenylated benzoylphloroglucinol derivatives comprising extracting a combined extract from dried and powdered twigs of the plant by using an organic solvent, followed by drying the combined extract under vacuum to obtain a petroleum ether-soluble part while the remaining matters are refluxed with preferably 80% EtOH which is further dried under vacuum to produce a residue which is suspended in water and extracted with EtOAc to obtain a EtOAc-soluble part, and isolating said derivatives from said petroleum ether-soluble part.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows chemical structures 1 and 2 for two novel compounds, garciesculentones A and C, isolated from Garcinia esculenta Y. H. Li.

FIG. 2 shows key correlations observed in the HMBC (H→C) NMR spectra of garciesculentones A and C (named as compounds 1 and 2, respectively).

FIG. 3 shows wound healing assay of garciesculentones A and C (named as compounds 1 and 2, respectively) on human esophageal carcinoma (ECA109) cells.

### DETAILED DESCRIPTION OF INVENTION

The present invention is not to be limited in scope by any of the specific embodiments described herein. The following embodiments are presented for exemplification only.

Garcinia esculenta Y. H. Li is the endemic plant of People's Republic of China, which is mainly distributed in the western and north-western part of Yunnan province. To date, there are still no reports on phytochemical and pharmacological research of this species. In the present invention, the chemical constituents of the twigs of G. esculenta Y. H. Li are found to contain potential anticancer lead compounds.

Compound Characterization

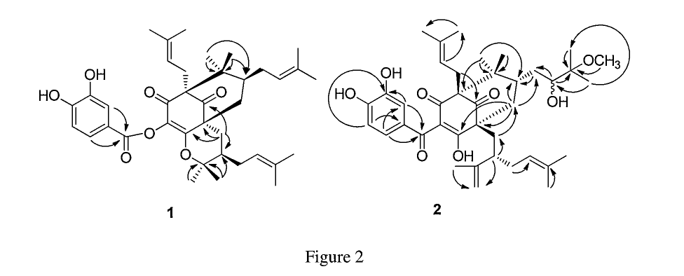

Garciesculentone A (named as compound 1) is obtained as a colorless gum. The ¹³C NMR spectrum of compound 1 shows characteristic resonances for six aromatic carbons and a bicyclic[3.3.1]nonane-2,4,9-trione moiety with three quaternary carbons (δ_{C}69.7, 47.0, and 52.7), one methine (δ_{C}47.0), one methylene (δ_{C}39.3), a nonconjugated ketone (δ_{C}206.6), and an enolized 1,3-diketone (δ_{C}192.7, 133.8, and 165.6). The HRESIMS shows an ion peak at m/z 617.3475 [M−H]⁻, giving the molecular formula C₃₈H₅₀O₇, differing from that of cambogin by 16 Da, corresponding to an additional oxygen. The NMR data (Table 1) obtained from 1D and 2D NMR spectra show close similarities to those of cambogin, with the exception of some differences of carbon signals due to one more oxygen atom in compound 1. The chemical shifts of C-3 and C-10 are at δ_{C}133.8 and 165.3 instead of δ_{C}124.7 and 192.3 in cambogin, respectively, which indicates that this additional oxygen atom is located between C-3 and C-10. This conclusion is supported by correlations of the proton signals at δ_{H}7.50 (1H, m) and 7.53 (1H, m) to C-10 (δ_{C}165.3). Other key HMBC correlations are shown in FIG. 2. In this manner, the planar structure of compound 1 is determined.

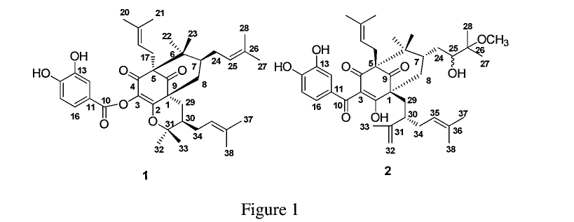

The relative configuration of compound 1 is deduced by the analysis of the ¹³C NMR and NOESY spectra. The ¹³C NMR chemical shift of C-7 at δ_{C}47.0 suggests that H-7 is α-oriented, or otherwise its signal would be located at δ_{C}41.0-44.0. The NOE correlations between CH₃-22/H-7 and CH₃-22/H-17 indicate that they are both α-oriented. The configuration of H-30 is deduced as being α-oriented by the NOE correlation of H-29/H-30. Similarly, CH₃-32 is suggested to be in the a-orientation by the NOE correlation of H-30/CH₃-32. Therefore, the structure of garciesculentone A (compound 1) is established as shown in FIG. 1.

Garciesculentone C (named as compound 2) is obtained as a yellow gum and shows a deprotonated molecular ion peak at m/z 649.3723 [M−H]⁻ in the HRESIMS, corresponding to the formula C₃₉H₅₃O₈ (calcd 649.3740). The ¹H NMR of compound 2 reveals the presence of one terminal double bond [δ_{H}4.46 (2H, brs)], two olefinic protons [δ_{H}5.04 (1H, m); δ_{H} 5.02 (1H, m)], one methoxy group [δ_{H}3.18 (3H, s)], and one 1,3,4-trisubstituted benzene ring [δ_{H}6.67 (1H, d, J=8.3 Hz), 6.93 (1H, dd, J=2.1 and 8.3 Hz), and 7.15 (1H, d, J=2.1 Hz)]. The ¹³C NMR and DEPT spectra of compound 2 (Table 1) disclosed a total of 39 carbon signals, corresponding to ten methyls, six methylenes, eight methines, and fifteen quaternary carbons. These NMR data (Table 1) are nearly identical with those of isogarcimultiflorone F, but shows one methoxy signal more [δ_{H}3.18 (3H, s), δ_{C}49.6]. This additional methoxy group is assigned at C-26 on the basis of the correlations of the methoxy protons at δ 3.18 to C-26 (δ_{C}78.5), from H-25 (δ_{H}3.27) to C-26 and C-24 (δ_{C}78.5), as well as from H-24 (δ_{H}1.48) to C-7 (δ_{C}42.9) in the HMBC spectrum. Detailed correlations of different groups in compound 2 is shown in FIG. 2.

TABLE 1
¹³C and ¹H NMR Data (100 and 400 MHz, J in Hz) of
Garciesculentone A and C (Compounds 1 and 2^{a})

| | garciesculentone A | | garciesculentone C | |
|---|---|---|---|---|
| | (compound 1)^{b} | | (compound 2)^{c} | |
| position | δ_{C} | δ_{H,} mult. (J in Hz) | δ_{C} | δ_{H,} mult. (J in Hz) |
| 1 | 52.7 | | 58.7 | |
| 2 | 165.6 | | 196.6 | |
| 3 | 133.8 | | 117.9 | |
| 4 | 192.7 | | 194.2 | |
| 5 | 69.7 | | 70.5 | |
| 6 | 47.0 | | 50.4 | |
| 7 | 47.0 | 1.57, m | 42.9 | 1.76, m |
| 8 | 39.3 | 2.37, brd (14.6); | 42.6 | 2.27, brd (13.9); |
| | | 1.99, m | | 2.08, m |
| 9 | 206.6 | | 210.7 | |
| 10 | 165.3 | | 195.3 | |
| 11 | 121.5 | | 129.4 | |
| 12 | 118.0 | 7.50, m | 117.3 | 7.15, d (2.1) |
| 13 | 146.3 | | 146.3 | |
| 14 | 152.3 | | 152.5 | |
| 15 | 116.1 | 6.85, d (8.7) | 115.1 | 6.67, d (8.3) |
| 16 | 124.4 | 7.53, m | 125.2 | 6.93, dd (2.1, 8.3) |
| 17 | 26.8 | 2.59, m; 2.51, m | 27.1 | 2.71, m; 2.57, m |
| 18 | 121.4 | 4.79, m | 121.3 | 5.04, m |
| 19 | 133.9 | | 135.8 | |
| 20 | 26.3 | 1.61, s | 26.5 | 1.72, s |
| 21 | 18.0 | 1.65, s | 18.3 | 1.69, s |
| 22 | 27.2 | 0.99, s | 27.7 | 1.01, s |
| 23 | 21.8 | 1.32, s | 23.1 | 1.14, s |
| 24 | 30.68^{d} | 2.47, m | 31.5 | 1.48, m; 1.40, m |
| 25 | 125.8 | 4.94, m | 75.4 | 3.27, m |
| 26 | 134.2 | | 78.5 | |
| 27 | 26.2 | 1.72, s | 21.2 | 1.00, s |
| 28 | 18.2 | 1.69, s | 20.8 | 1.02, s |
| 29 | 29.4 | 2.97, dd (3.52, 14.1) | 37.5 | 1.97, m |
| 30 | 44.3 | 1.46, m | 45.2 | 2.64, m |
| 31 | 88.4 | | 149.5 | |
| 32 | 21.8 | 1.32, s | 113.1 | 4.46, brs |
| 33 | 29.3 | 1.24, s | 18.2 | 1.58, s |
| 34 | 30.74^{d} | 2.10, m; 1.86, m | 33.4 | 2.03, m |
| 35 | 123.2 | 5.21, t (8.28) | 124.1 | 5.02, m |
| 36 | 134.6 | | 132.7 | |
| 37 | 26.0 | 1.77, s | 25.9 | 1.65, s |
| 38 | 18.3 | 1.65, s | 18.2 | 1.58, s |
| OCH₃ | | | 49.6 | 3.18, s |

^{a}Assignments are based on DEPT, HSQC, HMBC, and NOESY experiments; chemical shifts are given in ppm.
^{b}Measured in CD₃OD.
^{c}Measured in CD₃OD/0.1% TFA.
^{d}Data may be interchangeable.

The relative configuration of compound 2 is revealed by ¹³C NMR data and NOE correlations obtained from a NOESY spectrum. The ¹³C NMR chemical shift of C-7 at δ_{C}42.9 indicates that H-7 is fi-oriented, since the expected signal of H-7 in a-orientation would be between δ_{C}45.0 and 48.0. The C-23 methyl group and C-17 methylene group are determined to be both β-oriented from the correlations of CH₃-23/H-7, CH₃-22/H-24, and CH₃-23/H-17 found in the NOESY spectrum. Accordingly, the structure of garciesculentone C (compound 2) is established as shown in FIG. 1.

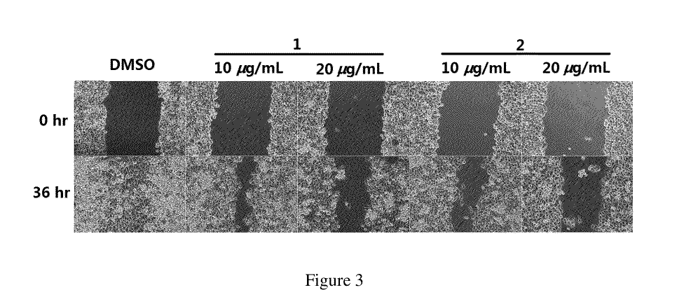

In the present invention, garciesculentones A and C (compounds 1 and 2) are evaluated for their cytotoxicity against two human hepatoma carcinoma (SMMC-7721 and HepG2) and two human breast adenocarcinoma cell lines (MCF-7 and MDA-MB-231) using MTT assay, palcitaxel is used as positive control. As shown in Table 2, compounds 1 and 2 exhibit significant cytotoxicity against one of the tested human cancer cell lines for which IC₅₀ values are below or near 10 μM. In practice, an effective concentration of the tested compound to kill a target cancer cell in vitro is normally about double of the corresponding IC₅₀ value. In addition, wound healing assay is conducted to test whether two novel compounds could affect cell motility. Maintained in completed medium, a confluent monolayer of ECA109 cells is scratched to form a wound and incubated in the absence or presence of tested compounds at the concentrations of 10 and 20 μg/mL (Compounds tested at the highest concentration of 20 μg/mL do not alter the viability of ECA109 cells as compared to that of controls). The cells migrated into the wound area by 36 hours to such an extent that the wound edges are indistinguishable in control group, while compounds 1 and 2 treated cells do not migrate into and completely close the wound (FIG. 3), indicating that both compounds have antimigration effect on ECA109 cells in the absence of cytotoxicity.

TABLE 2
Cytotoxicity of Novel Compounds against Four Cancer Cell Lines ^{a}

| Compound | SMMC-7721 | HepG 2 | MCF-7 | MDA-MB-231 |
|---|---|---|---|---|
| 1 | 10.8 | 43.2 | 20.3 | 16.4 |
| 2 | 17.5 | 21.9 | 11.1 | 21.5 |
| Palcitaxel^{b} | 9.1 × 10⁻³ | 4.6 × 10⁻³ | 1.5 × 10⁻³ | 8.2 × 10⁻³ |

^{a} Results are expressed as IC₅₀ values in μM.
^{b}Positive control.

Experimental Section

General Experimental Procedures. Optical rotations were measured with a JASCO P-1020 polarimeter. Ultraviolet absorption spectra were recorded on a UV-2401 PC spectrophotometer. IR spectra were obtained from a Bio-Rad FtS-135 spectrometer. NMR spectra were measured on a Bruker AV-400 spectrometer with TMS as the internal standard. Mass spectrometry was performed on a Waters Q-TOF Premier instrument (Micromass MS Technologies, Manchester, UK) spectrometer, with an electrospray ion source (Waters, Milford, Mass.) connected to a lock-mass apparatus performing a real-time calibration correction. Column chromatography was performed with CHP20P MCI gel (75-150 ,um, Mitsubishi Chemical Coparation, Japan), silica gel (200-300 mesh, Qingdao Haiyang Chemical Co., Ltd.), Sephadex LH-20 (GE Healthcare Bio-Sciences AB, Sweden), and reversed-phase C18 silica gel (50 μm, YMC, Kyoto, Japan). Precoated TLC sheets of silica gel 60 GF254 (Qingdao Haiyang Chemical Co., Ltd.) were used. A waters 2535 Series machine equipped with a Xbridge C₁₈ column (4.6×250 mm, 5 μm) was used for HPLC analysis, and preparative Xbridge Prep C₁₈ OBD column (19×250 mm, 5 μm) was used in sample preparation. Paclitaxel was purchased from Sigma-Aldrich Trading Co. Ltd. (Shanghai, People's Republic of China).

Plant Material. The twigs of Garcinia esculenta Y. H. Li were collected in Nujiang, Yunnan Province, People's Republic of China, in August 2010. The plant material was identified by Prof. Yuanchuan Zhou, Yunnan University of Traditional Chinese Medicine. A voucher specimen (Herbarium No. 20100801) has been deposited at the Innovative Research Laboratory of TCM, Shanghai University of Traditional Chinese Medicine.

Extraction and Isolation. Air-dried and powdered twigs of the plant (4 kg) were extracted with petroleum ether (5×20 L, each two days). The combined extracts were evaporated to dryness under vacuum to gain the petroleum ether-soluble part (fraction I, 40 g). The remaining materials were refluxed with 80% EtOH (v/v, 5×20 L). The combined extracts were evaporated to dryness under vacuum and the residue was suspended in H₂O (5 L) and extracted with EtOAc (5×5 L) to obtain fractions II (50 g, the EtOAc-soluble part) and III (the remainin H₂O part), respectively. The remaining materials were refluxed with distilled water (5×20 L) to gain the H₂O-soluble part (fraction IV). Fractions I and II were shown to possess the significant cytotoxic activities against four human cancer cell lines.

Fraction I (37 g) was chromatographed on a silica gel column chromatography (CC) using a gradient of petroleum ether-EtOAc (100:0 to 50:50, v/v), and yielded fifteen fractions (IA-IO) based on the TLC profiles. Fraction IL was chromatographed separately on MCI gel eluted with 90% and 100% EtOH, successively, to afford two subfractions (IL1 and IL2), respectively. Fraction ILle was further separated by preparative HPLC (MeOH-MeCN-H₂O, 27.2:40.8:32, with 0.1% formic acid in H₂O, 20 mL/min) to give subfractions IL1e1-IL1e7. Compound 2 (9 mg) was finally obtained by preparative HPLC from fraction IL1e3. Fraction IL1f was separated by preparative HPLC (20 mL/min), eluting with CH₃OH-H₂O (12:88, with 0.1% formic acid in H₂O) to give subfractions IL1f1-IL1f5. Fraction IL1f3 was finally purified by preparative HPLC (MeOH—MeCN—H₂O, 30:45:25 with 0.1% formic acid in H₂O, 20 mL/min) to yield compound 1 (8 mg).

Garciesculentone A (compound 1): colorless gum; [α]²⁰_{D}−116.5 (c 0.06, MeOH); UV (MeOH) λₘₐₓ (logε) 268 (4.26) nm; IR (KBr) νₘₐₓ3423, 2976, 2927, 2858, 1738, 1699, 1660, 1608, 1523, 1444, 1373, 1346, 1292, 1197, 1122, 1089, 960, 825, 756 cm⁻¹; ¹H NMR (CD₃OD, 400 MH_{z}) data, see Table 1; ¹³C NMR (CD₃OD, 100 MH_{z}) data, see Table 1; HRESIMS 617.3475 [M−H]⁻ (calcd for C₃₈H₄₉O₇, 617.3478).

Garciesculentone C (compound 2): yellow gum; [α]²⁰_{D}−24.9 (c 0.08, MeOH); UV (MeOH) λₘₐₓ (logε) 279 (4.11) nm; IR (KBr) νₘₐₓ 3424, 2970, 2928, 1686, 1438, 1376, 1291, 1207, 1143, 893, 804, 724 cm⁻¹; ¹H NMR (CD₃OD, 400 MH_{z}) data, see Table 1; ¹³C NMR (CD₃OD, 100 MH_{z}) data, see Table 1; HRESIMS m/z 649.3723 [M−H]⁻ (calcd for C₃₉H₅₃O₈, 649.3740).

Cytotoxicity Assay. Cytotoxic activities of all isolates were evaluated by MTT assay using SMMC-7721 (human hepatocellular carcinoma), HepG2 (human hepatocellular carcinoma), MCF-7 (human breast adenocarcinoma), and MDA-MB-231 (human breast adenocarcinoma). Paclitaxel was used as positive control. The detailed methodology for cytotoxicity assay has already been described in a previous report in Xia, Z.-X.; Zhang, D.-D.; Liang, S.; Lao, Y.-Z.; Zhang, H.; Tan, H.-S.; Chen, S.-L.; Wang, X.-H.; Xu, H.-X., Bioassay-Guided Isolation of Prenylated Xanthones and Polycyclic Acylphloroglucinols from the Leaves of Garcinia nujiangensis. Journal of Natural Products 2012.

Wound healing assay. A wound healing assay was performed by using a modified procedure described previously in Jones, J. I.; Gockerman, A.; Busby, W. H., Jr.; Wright, G.; Clemmons, D. R., Insulin-like growth factor binding protein 1 stimulates cell migration and binds to the alpha 5 beta 1 integrin by means of its Arg-Gly-Asp sequence. Proc Nall Acad Sci USA 1993, 90 (22), 10553-7. Briefly, cells were plated in 24 wells plate at 70% confluence in conditioned medium. 24 hours after seeding the monolayers were wounded by scoring with a sterile plastic 20 μL micropipette tip, then rinsed several times with media to remove cell debris and then incubated in completed medium in the absence or presence of tested compounds (10 and 20 μg/mL) for various periods of time up to 36 hours. Cell migration into the wound surface was monitored by Olympus 1X71 microscopy and digitally photographed.

### INDUSTRIAL APPLICATION

The present invention discloses new chemical entities of Garcinia esculenta Y. H. Li having cytotoxicity against a panel of human cancer cell lines. In particular, The present invention relates to two novel polyprenylated benzoylphloroglucinol derivatives, garciesculentones A and C (compounds 1 and 2), which exhibit cytotoxicity and antimigration effect against various human cancer cell lines, thus, can be developed as anticancer drugs.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A composition for treating cancer comprising compounds with at least one of the following chemical structures extracted from a natural plant:

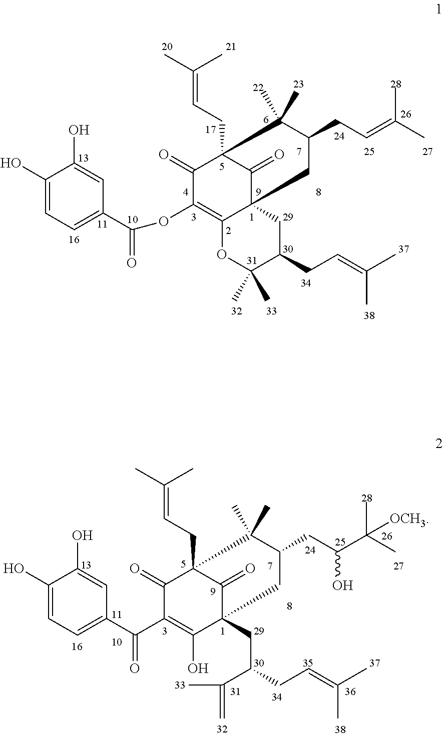

2. The composition according to claim 1 wherein said compounds comprise polyprenylated benzoylphloroglucinol derivatives.

3. The composition according to claim 2 wherein said polyprenylated benzoylphloroglucinol derivatives comprise Garciesculentone A and/or Garciesculentone C.

4. The composition according to claim 1 wherein the natural plant is from the genus Garcinia.

5. The composition according to claim 4 wherein the subspecies of genus Garcinia is Garcinia esculenta Y. H. Li.

6. The composition according to claim 1 wherein said compounds are extracted from twigs of said plant.

7. The composition according to claim 1 wherein the cancer treated comprises hepatoma carcinoma, breast adenocarcinoma and esophageal carcinoma.

8. A method for preparing the composition of claim 1 comprising extracting a combined extract from dried and powdered twigs of the plant by using an organic solvent, followed by drying the combined extract under vacuum to obtain a petroleum ether-soluble part while the remaining matters are refluxed with preferably 80% EtOH which is further dried under vacuum to produce a residue which is suspended in water and extracted with EtOAc to obtain a EtOAc-soluble part, and isolating said compounds from said petroleum ether-soluble part.

9. A method for treating cancer comprising administering the composition of claim 1 to a subject in needs thereof.
